(19) United States
(12) Patent Application Publication
(10) Pub. No.: US 2020/0179652 A1
(43) Pub. Date: Jun. 11, 2020

(54) Alternative Fluid-Driven Articulation Architecture for Catheters and Other Uses

(71) Applicant: Project Moray, Inc., Belmont, CA (US)

(72) Inventors: Ronald G. Williams, Menlo Park, CA (US); Keith Phillip Laby, Oakland, CA (US); Mark D. Barrish, Belmont, CA (US); Tim Williams, Palo Alto, CA (US)

(73) Assignee: Project Moray, Inc., Belmont, CA (US)

(21) Appl. No.: 16/790,568

(22) Filed: Feb. 13, 2020

Related U.S. Application Data
(63) Continuation of application No. PCT/US2018/046315, filed on Aug. 10, 2018.
(60) Provisional application No. 62/545,335, filed on Aug. 14, 2017.

(51) Int. Cl. A61M 25/01; A61M 25/00

(52) U.S. Cl. CPC A61M 25/0155; A61M 25/0045; A61M 2025/09008; A61M 25/005; A61M 25/0138; A61M 25/0074

(57) ABSTRACT

Devices, systems, and methods for articulating elongate flexible structures such as catheters optionally include an array of fluid-expandable bodies such as balloons. The array can be formed using separate strings of balloons formed along single-lumen balloon tube material. The balloon strings can be twisted together to form a multi-channel bundle, or the balloon strings may be circumferentially separated, each extending axially. Regardless, the balloons along a common lumen may be aligned so as to bend the catheter in a desired lateral direction. The fluid-expandable bodies may include an elastomeric bladder with a fiber braid so that inflation of the bladder shortens the assembly and applies axial tension to articulate the catheter. The elongate flexible structures may be pre-biased so as to form a bend when in a relaxed configuration, with the structures being articulatable from the bend.

### CROSS REFERENCE TO RELATED APPLICATIONS

The present application is a Continuation of PCT/US2018/046315 filed Aug. 10, 2018; which claims the benefit of U.S. Provisional Appln No. 62/545,335 filed Aug. 14, 2017, the full disclosures which are incorporated herein by reference in their entirety for all purposes.

### FIELD OF THE INVENTION

In general, the present invention provides improved devices, systems, and methods for articulation of elongate flexible bodies such as catheters, borescopes, continuum robotic manipulators, and the like. In some exemplary embodiments, the invention provides articulated structures and methods for altering the resting shape (and particularly the axial bending characteristics) of catheters using a fluid-driven articulation balloon array in which subsets of balloons in the array are formed integrally from a substrate material such as a tube of material suitable for blowing balloons. The substrate often defines an inflation fluid lumen for the balloon subset, and other balloon array subsets (and their associated inflation lumens) will often be formed separately. In exemplary embodiments, the elongate flexible body can be biased so as to form (and be articulated from) a desired axial series of bends when in a relaxed configuration, with or without the separate balloon subsets. Still further embodiments are described below

### BACKGROUND OF THE INVENTION

Diagnosing and treating disease often involve accessing internal tissues of the human body, and open surgery is often the most straightforward approach for gaining access to internal tissues. Although open surgical techniques have been highly successful, they can impose significant trauma to collateral tissues.

To help avoid the trauma associated with open surgery, a number of minimally invasive surgical access and treatment technologies have been developed, including elongate flexible catheter structures that can be advanced along the network of blood vessel lumens extending throughout the body. While generally limiting trauma to the patient, catheter-based endoluminal therapies can be very challenging. Alternative minimally invasive surgical technologies include robotic surgery, and robotic systems for manipulation of flexible catheter bodies from outside the patient have also previously been proposed. Some of those prior robotic catheter systems have met with challenges, in-part because of the difficulties in accurately controlling catheters using pull-wires. While the potential improvements to surgical accuracy make these efforts alluring, the capital equipment costs and overall burden to the healthcare system of these large, specialized systems is a concern.

A new technology for controlling the shape of catheters has recently been proposed which may present significant advantages over pull-wires and other known catheter articulation systems. As more fully explained in US Patent Publication No. US20160279388, entitled “Articulation Systems, Devices, and Methods for Catheters and Other Uses,” published on Sep. 29, 2016 (assigned to the assignee of the subject application and the full disclosure of which is incorporated herein by reference), an articulation balloon array can include subsets of balloons that can be inflated to selectively bend, elongate, or stiffen segments of a catheter. These articulation systems can direct pressure from a simple fluid source (such as a pre-pressurized canister that remains outside) a patient toward a subset of articulation balloons disposed along the segment(s) so as to induce a desired change in shape. The pressurized inflation fluid can be transmitted to and from the subsets of balloons via, for example, ports selectively laser-drilled into a series of channels in a simple multi-lumen extrusion. A tube of balloon material can be placed over the balloon extrusion and sealed over the ports, with the ports providing fluid communication between the subsets of the balloons and their associated lumens in the extrusion, providing catheter control beyond what was previously available, often without having to resort to a complex robotic gantry, without pull-wires, and even without motors. Hence, these new fluid-driven catheter systems appear to provide significant advantages.

Despite the advantages of the newly proposed fluid-driven catheter system, as with all successes, still further improvements and alternatives would be desirable. In general, it would be beneficial to provide further improved medical systems, devices, and methods, as well as to provide alternative architectures for articulating flexible bodies via fluid pressure. More specifically, it may be beneficial to facilitate balloon articulation of catheters and other devices without relying on intermittent sealing of a balloon tube over a multi-lumen extrusion. It may also be beneficial to provide elongate flexible structures that are configured to have an articulation workspace that is tailored to a particular anatomical or other workspace, such that a range of motion of the structure is not axisymmetric about the elongate axis.

### BRIEF SUMMARY OF THE INVENTION

The present invention generally provides improved devices, systems, and methods for articulating elongate flexible structures such as catheters, borescopes, continuum robotic manipulators, and the like. The elongate flexible structures described herein will often include an array of fluid-expandable bodies such as balloons. Optionally, the arrays can be formed using separate strings of balloons, each formed along a single-lumen tube of balloon material. The balloon strings can be twisted together to form a multi-channel bundle, or the balloon strings may optionally be circumferentially separated about the articulated structure, each extending axially with the balloons aligned so as to bend the catheter or other structure in a desired lateral direction. Alternative embodiments make use of fluid-expandable bodies that include an elastomeric bladder coaxial with a fiber braid or the like, with the fibers being configured so that inflation of the bladder axially shortens the assembly such that the string applies axial tension to articulate the catheter. Still further alternative embodiments include elongate flexible structures that are pre-biased so as to form a bend when in a relaxed configuration, with the structures typically being articulatable from the bend using the balloons of an articulation balloon array.

In a first aspect, the invention provides an articulation system comprising an elongate flexible structure having a proximal end and a distal end with an axis therebetween. At least one articulated segment extends along the axis, and an array of fluid-expandable bodies are distributed along the at least one articulated segment. The array comprises a first array string and a second array string. The first array string includes a first subset of fluid-expandable bodies offset from the axis so as to laterally articulate the structure with a first lateral bend when inflated, and a first inflation tube having a first lumen in fluid communication with the expandable bodies of the first subset. The second array string is separate from the first string along the at least one articulated segment and includes a second subset of fluid-expandable bodies offset from the axis so as to laterally deflect the structure with a second bend offset from the first bend when inflated, and a second inflation tube having a second lumen in fluid communication with the bodies of the second subset.

As a general feature, the separate array strings can each be formed as a linear sub-array, the expandable bodies and lumen of the string optionally being integral structures formed from a common substrate material such as by locally expanding a diameter of a polymer tube material at selected locations to form a series of balloons separated by discontinuous segments of the unexpanded (or less expanded) lumen. Only the inflation fluid for a particular sub-array of expandable bodies may flow through the lumen of that sub-array, rather than (for example) having inflation fluid for multiple sub-arrays being directed along separate parallel channels of a multi-lumen extrusion or the like. When fluid pressure is transmitted through the lumen of the string, all the expandable bodies of that string may be inflated by and to that pressure (once equilibrium is achieved). Processing of the formed string components to selectively contain fluid flow (such as laser drilling of a multi-lumen so as to direct fluid from a particular channel to some but not all of the expandable bodies of the string, or boding balloon material over a multi-lumen) may be avoided. Preferably, the expandable bodies of the first subset are formed from material of the first tube so that inflation fluid flows through a first port. The first body is in fluid communication to the other expandable bodies via a second port of the first body, and the other expandable bodies being in fluid communication, in series, to each other via other associated ports, the ports being substantially inexpansible, the port cross-section typically not expanding by more than 10% throughout an operating inflation range during use. Pairs of ports in all but the last expandable body direct the inflation fluid to inflate the expandable bodies in series. Discontinuous segments of the lumen for the string may provide fluid communication between the ports.

Optionally, the first and second inflation tubes can be twisted together in an inflation tube bundle, with the bundle often winding helically along the segment(s). The balloons may take the form of offset balloons (relative to the inflation tube) and the twisting of the tubes can be concentrated primarily between the balloons so that the balloons are in alignment along a side of the bundle (such as the radially inward side or radially outward side of a helical bundle). The spacing of the balloons within their associated array strings and the helical arc-lengths along the path of the twisted bundles will often correspond, for example, so that the expandable bodies of one string are aligned along one side of the structure, such that inflation of that string via its common lumen induces bending in the associated bending orientation. The expandable bodies of another string in the bundle can be aligned along another side for bending in a transverse orientation, with three or four strings being included in the bundle having three or four associated circumferentially offset subsets of expandable bodies.

In alternative arrangements, as the subsets of different array strings may perform differing articulation functions, the strings may be separated along at least an active articulation-inducing portion of their length. For example, the first and second array strings may be circumferentially separated (rather than being twisted together), at least along a first articulated segment. The first articulated segment may, for example, have a working lumen extending along the axis, and the articulated system may include a plurality of annular bodies distributed along the axis. Each annular body can be disposed around the working lumen, and a resiliently flexible inner sheath or other axial support may maintain separation between adjacent annular bodies (such as by boding of the annular bodies to the sheath or the like). The annular bodies can have circumferentially offset channels receiving the tubes of the array strings axially therethrough. The array strings can optionally engage the annular bodies so that axial articulation forces induced by inflation of the expandable bodies are transmitted to the annular bodies to induce curved articulation along the first segment. The flexible structure often includes a second articulated segment axially offset from the first articulated segment, the second articulated segment having a plurality of axially separated annular bodies and a plurality of array strings configured to imposed articulation forces thereto so as to facilitate curved articulation of the second segment independent of the first segment. The strings for this second segment may also be separated circumferentially along at least the second segment; additional strings may be provided for one or more additional segments. The strings can be adjacent to each other (or even integrated into a common multi-channel structure) as they run proximally from their associated articulation segment. In general, the expandable bodies may optionally comprise balloons, the balloons often being disposed between adjacent annular bodies. The balloons can have opposed ends, each adjacent an associated inflation fluid port. The balloon ends can engage and apply compressive forces against the adjacent annular bodies so as to urge the adjacent annular bodies apart when the balloons are inflated.

Still further alternative architectures may be provide, for example, with the expandable bodies having ends affixed to adjacent annular bodies and including radially expandable bladders and fibers extending circumferentially about the bladders. Such fibers can be loaded in tension and urge the adjacent annular bodies together when the expandable bodies are inflated. These and other tension-inducing expandable bodies may have structures and force generation characteristics associated with known McKibben muscle actuators and related variants, and will often have lengths (between the ends) greater than their inflated diameters during use. Other expandable bodies may have related structures (including resiliently inflatable bladders supported by radially oriented fibers) but may have larger diameters than lengths in at least some configurations during use, and may induce compressive bending loads against the adjacent annular bodies.

Optionally, the first and second subsets are axially separated and are disposed along first and second axial articulation segments, respectively. In these and other embodiment, the axis along the first articulation segment can define a bend when the structure is in a relaxed configuration. Preferably, the first articulation segment is deformable toward an axially straight configuration for insertion into a patient. To align the range of motion of the articulated structure with a target workspace, variable inflation of the expandable bodies will often variably articulate the first segment within a range of motion from the bend with a first degree of freedom, rather than having the range of motion being centered about an axially straightened configuration. A plurality of other articulation segments can define associated axial bends when in the relaxed configuration, and can be articulatable from the associated bends with associated degrees of freedom. Advantageously, the degrees of freedom can be arranged so as to allow independent translation and rotation of the structure, optionally with one, some, or even all of the segment having only one associated degree of freedom. The bend in the relaxed state can be provided by a number of alternative features, some of which can be quite simple. For example, when the fist articulated segment comprise a helical coil spring having an axial series of loops with axial spaces therebetween, with the expandable bodies of the first subset being disposed within the spaces of the spring, an asymmetrical series of spacers can be provided and positioned between the loops and expandable bodies so as to urge the first articulated segment toward the bend when the structure is in the relaxed configuration. The spacers may comprise arc-segments of annular or helical structures (such as 90 degree or 120 degree arc segments of a washer or ribbon spring), and positioning of spacers of suitable thickness at appropriate circumferential orientations along different segment can provide nominal bends of differing orientations and radii along the segments.

As optional general features, the first and second subsets can each include three or more fluid expandable bodies. A third array string having a third subset of fluid expandable bodies may be disposed along the at least one articulated segment so as to articulate the structure laterally along three lateral bending orientations. Advantageously, the structure can comprise a frame and a polymer matrix. the array strings can be embedded in the polymer matrix on or within the frame so as to maintain alignment of the subsets of expandable bodies relative to the axis.

In another aspect, the invention provides an articulation system comprising an elongate flexible structure having a proximal end and a distal end with an axis therebetween. An articulated segment typically extends along the axis. A first subset of balloons may be offset from the axis so that a first inflation fluid pressure within the first subset of balloons laterally deflects the body with a first lateral bending orientation when the balloons are inflated. The first subset of balloons can include a first balloon, a last balloon, and a plurality of intermediate balloons. A first lumen may be disposed within the flexible structure, the first lumen being discontinuous such that the first lumen extends distally to a first port of the first balloon of the first subset, and from a second port of the first balloon of the first subset to an adjacent port of an intermediate balloon of the first subset. The first lumen can sequentially connect the balloons of the first subset (optionally via a series of additional lumen segments) such that the first inflation fluid pressure within the last balloon of the first subset is transmitted by fluid communication through the first balloon of the first subset and the intermediate balloons of the first subset. A second subset of balloons may be offset from the axis so that inflation fluid pressure within the second subset of balloons laterally deflects the body along a second lateral bending orientation. The second subset of balloons can include a first balloon, a last balloon, and a plurality of intermediate balloons. A second lumen may be disposed within the body, and the second lumen can be discontinuous such that the second lumen extends distally to a first port of the first balloon of the second subset, and can extend from a second port of the first balloon of the second subset to an adjacent port of an intermediate balloon of the second subset. The second lumen can sequentially connect the balloons of the second subset such that the second inflation fluid pressure within the last balloon of the second subset is transmitted by fluid communication through the first balloon of the second subset and the intermediate balloons of the second subset.

In another aspect, the invention provides an articulation system comprising an elongate structure having a proximal end and a distal end with an axis therebetween. A first (and optionally a second) articulated segment extends along the axis. An array of fluid-expandable bodies are distributed along the first articulated segment, the array comprising a first subset of fluid-expandable bodies offset from the axis so as to laterally articulate the structure along a first lateral bending orientation. The axis along the first segment defines a bend when the structure is in a relaxed configuration. The first articulation segment is resiliently deformable toward an axially straight configuration for insertion into a patient. The structure and array are configured so that variable inflation of the first subset variably articulates the first segment in a range of motion from the bend. Optionally, a second subset of fluid-expandable bodies are disposed along the second segment and offset from the axis so as to laterally deflect the structure along a second lateral bending orientation.

In yet another aspect, the invention provides an articulation system comprising an elongate structure having a proximal end and a distal end with an axis therebetween. A first (and optionally a second) articulated segment extends along the axis. An array of fluid-expandable bodies are distributed along the first articulated segment, the array comprising a first subset of fluid-expandable bodies offset from the axis so as to laterally articulate the structure along a first lateral bending orientation. A second subset of fluid-expandable bodies are disposed along the second segment and offset from the axis so as to laterally deflect the structure along a second lateral bending orientation.

In yet another aspect, the invention provides an articulation system comprising an elongate structure having a proximal end and a distal end with an axis therebetween. First, second, and third articulated segments extend along the axis. An array of fluid-expandable bodies is distributed along the first, second, and third articulated segments. The array comprises a first subset of fluid-expandable bodies offset from the axis so as to laterally articulate the first articulated segment along a first lateral bending orientation, a second subset of fluid-expandable bodies offset from the axis so as to laterally deflect the second articulated segment along a second lateral bending orientation, and a third subset of fluid-expandable bodies offset from the axis so as to laterally deflect the third articulated segment. Variable inflation of the array can variably articulate the elongate structure in at least three degrees of freedom. Preferably, the array comprises three or more subsets along three or more associated segments, and each segment has a single associated subset of the array. Each segment (with its associated subset) can be configured so as to provide lateral bending in a single associated bending orientation, and the array can provide movement of the distal end with from three to six degrees of freedom.

### BRIEF DESCRIPTION OF THE DRAWINGS

FIG. 1 is a simplified perspective view of a medical procedure in which a physician can input commands into a catheter system so that a catheter is articulated using systems and devices described herein.

FIGS. 2A-2C schematically illustrates a catheter having a distal portion with an axial series of articulated segments supporting a prosthetic mitral valve, and show how the segments articulate so as to change the orientation and location of the valve.

FIGS. 3A-3C schematically illustrate input command movements to change the orientation and location of the valve, with the input commands corresponding to the movements of the valve so as to provide intuitive catheter control.

FIG. 4 is a partially see-through perspective view of an exemplary fluid drive manifold system for articulating a balloon array so as to control the shape of a valve delivery catheter or other elongate flexible body.

FIG. 5 is a simplified schematic illustration of components of a helical balloon assembly, showing how an extruded multi-lumen shaft can be assembled to provide fluid to laterally aligned subsets of the balloons.

FIGS. 6A-6C schematically illustrate helical balloon assemblies supported by flat springs and embedded in an elastomeric polymer matrix, and show how selective inflation of subsets of the balloons can elongate and laterally articulate the assemblies.

FIGS. 7 and 8 are cross-sections schematically illustrating a polymer dip coat supporting helical balloon assemblies with the balloons nominally inflated and fully inflated, respectively.

FIGS. 9-11 are cross-sections schematically illustrating a dip-coated helical balloon assembly having a flat spring between axially adjacent balloons in an uninflated state, a nominally inflated state, and a fully inflated state, respectively, with the dip coating comprising a soft elastomeric matrix.

FIG. 12 is a cross-section schematically illustrating yet another alternative dip-coated helical balloon assembly embedded within a relatively soft polymer matrix, with support coils disposed radially inward and outward of the balloon assemblies and dip-coated in a different, relatively hard polymer matrix.

FIGS. 13A-13E schematically illustrate frame systems having axially opposed elongation and contraction balloons for locally elongating and bending a catheter or other elongate flexible body.

FIGS. 14A-14E schematically illustrate frame systems having axially opposed elongation and contraction balloons similar to those of FIGS. 13A-13E, with the frames comprising helical structures.

FIG. 15 is a cross-section schematically illustrating an elongation-contraction frame similar to those of FIGS. 13A-14E, showing a soft elastomeric polymer matrix supporting balloon assemblies within the frames.

FIGS. 16A-16C are perspective views of a balloon string having a single subset of offset balloons in fluid communication with a single inflation lumen, a bundle of three single-lumen balloon strings twisted together, and a balloon string having pre-bent balloons, respectively.

FIGS. 17A-17H are perspective and side views of an alternative ring-frame articulated structure in which six circumferentially offset balloon strings extend axially through channels in annular bodies, and in which selective inflation of the balloon strings locally separate adjacent features of the annular bodies so as to elongate and/or bend the structure.

FIGS. 18A-18D are perspective views of an alternative ring-frame articulated structure in which four circumferentially separated balloon strings each have balloons disposed between adjacent ring frames with ends of the balloons pushing the adjacent ring frames apart when inflated, along with selected components of the articulated structure.

FIGS. 19A-19D schematically illustrate another alternative ring-frame articulated structure in which four circumferentially separated balloon strings each have axially compressible balloons that separate the balloons when inflated.

FIGS. 20A and 20B are a schematic exploded view and a perspective view, respectively, of a ring frame articulation structure having four array strings of fluid-expandable bodies, in which the expandable bodies include an elastomeric bladder and a braided fiber sheath that imposes an axial tension between adjacent ring-frames when the bladder is inflated.

FIGS. 21A-21L show an articulated frame comprising a helical spring with spacers between loops of the spring and actuation balloons so as to bias the frame toward a bent configuration, along with perspective and side views showing an axial series of one degree-of-freedom articulated segments can move an end of the frame to a desired position and orientation.

### DETAILED DESCRIPTION OF THE INVENTION

The present invention generally provides fluid control devices, systems, and methods that are particularly useful for articulating catheters and other elongate flexible structures. The structures described herein are particularly well suited for catheter-based therapies, including for cardiovascular procedures such as those in the growing field of structural heart repair, as well as in the broader field of interventional cardiology. Alternative applications may include use in steerable supports of image acquisition devices such as for trans-esophageal echocardiography (TEE) and other ultrasound techniques, endoscopy, and the like. The structures described herein will often find applications for diagnosing or treating the disease states of or adjacent to the cardiovascular system, the alimentary tract, the airways, the urogenital system, and/or other lumen systems of a patient body. Other medical tools making use of the articulation systems described herein may be configured for endoscopic procedures, or even for open surgical procedures, such as for supporting, moving and aligning image capture devices, other sensor systems, or energy delivery tools, for tissue retraction or support, for therapeutic tissue remodeling tools, or the like. Alternative elongate flexible bodies that include the articulation technologies described herein may find applications in industrial applications (such as for electronic device assembly or test equipment, for orienting and positioning image acquisition devices, or the like). Still further elongate articulatable devices embodying the techniques described herein may be configured for use in consumer products, for retail applications, for entertainment, or the like, and wherever it is desirable to provide simple articulated assemblies with multiple degrees of freedom without having to resort to complex rigid linkages.

Embodiments provided herein may use balloon-like structures to effect articulation of the elongate catheter or other body. The term “articulation balloon” may be used to refer to a component which expands on inflation with a fluid and is arranged so that on expansion the primary effect is to cause articulation of the elongate body. Note that this use of such a structure is contrasted with a conventional interventional balloon whose primary effect on expansion is to cause substantial radially outward expansion from the outer profile of the overall device, for example to dilate or occlude or anchor in a vessel in which the device is located. Independently, articulated medial structures described herein will often have an articulated distal portion, and an unarticulated proximal portion, which may significantly simplify initial advancement of the structure into a patient using standard catheterization techniques.

The catheter bodies (and many of the other elongate flexible bodies that benefit from the inventions described herein) will often be described herein as having or defining an axis, such that the axis extends along the elongate length of the body. As the bodies are flexible, the local orientation of this axis may vary along the length of the body, and while the axis will often be a central axis defined at or near a center of a cross-section of the body, eccentric axes near an outer surface of the body might also be used. It should be understood, for example, that an elongate structure that extends “along an axis” may have its longest dimension extending in an orientation that has a significant axial component, but the length of that structure need not be precisely parallel to the axis. Similarly, an elongate structure that extends “primarily along the axis” and the like will generally have a length that extends along an orientation that has a greater axial component than components in other orientations orthogonal to the axis. Other orientations may be defined relative to the axis of the body, including orientations that are transvers to the axis (which will encompass orientation that generally extend across the axis, but need not be orthogonal to the axis), orientations that are lateral to the axis (which will encompass orientations that have a significant radial component relative to the axis), orientations that are circumferential relative to the axis (which will encompass orientations that extend around the axis), and the like. The orientations of surfaces may be described herein by reference to the normal of the surface extending away from the structure underlying the surface. As an example, in a simple, solid cylindrical body that has an axis that extends from a proximal end of the body to the distal end of the body, the distal-most end of the body may be described as being distally oriented, the proximal end may be described as being proximally oriented, and the surface between the proximal and distal ends may be described as being radially oriented. As another example, an elongate helical structure extending axially around the above cylindrical body, with the helical structure comprising a wire with a square cross section wrapped around the cylinder at a 20 degree angle, might be described herein as having two opposed axial surfaces (with one being primarily proximally oriented, one being primarily distally oriented). The outermost surface of that wire might be described as being oriented exactly radially outwardly, while the opposed inner surface of the wire might be described as being oriented radially inwardly, and so forth.

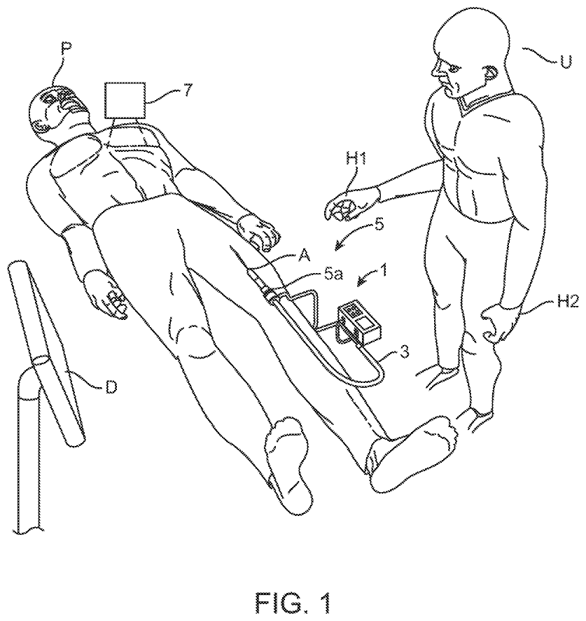

Referring first to FIG. 1, a first exemplary catheter system 1 and method for its use are shown. A physician or other system user U interacts with catheter system 1 so as to perform a therapeutic and/or diagnostic procedure on a patient P, with at least a portion of the procedure being performed by advancing a catheter 3 into a body lumen and aligning an end portion of the catheter with a target tissue of the patient. More specifically, a distal end of catheter 3 is inserted into the patient through an access site A, and is advanced through one of the lumen systems of the body (typically the vasculature network) while user U guides the catheter with reference to images of the catheter and the tissues of the body obtained by a remote imaging system.

Exemplary catheter system 1 will often be introduced into patient P through one of the major blood vessels of the leg, arm, neck, or the like. A variety of known vascular access techniques may also be used, or the system may alternatively be inserted through a body orifice or otherwise enter into any of a number of alternative body lumens. The imaging system will generally include an image capture system 7 for acquiring the remote image data and a display D for presenting images of the internal tissues and adjacent catheter system components. Suitable imaging modalities may include fluoroscopy, computed tomography, magnetic resonance imaging, ultrasonography, combinations of two or more of these, or others.

Catheter 3 may be used by user U in different modes during a single procedure. More specifically, at least a portion of the distal advancement of catheter 3 within the patient may be performed in a manual mode, with system user U manually manipulating the exposed proximal portion of the catheter relative to the patient using hands H1, H2. In addition to such a manual movement mode, catheter system 1 may also have a 3-D automated movement mode using computer controlled articulation of at least a portion of the length of catheter 3 disposed within the body of the patient to change the shape of the catheter portion, often to advance or position the distal end of the catheter. Movement of the distal end of the catheter within the body will often be provided per real-time or near real-time movement commands input by user U. Still further modes of operation of system 1 may also be implemented, including concurrent manual manipulation with automated articulation, for example, with user U manually advancing the proximal shaft through access site A while computer-controlled lateral deflections and/or changes in stiffness over a distal portion of the catheter help the distal end follow a desired path or reduce resistance to the axial movement. Additional details regarding modes of use of catheter 3 can be found in US Patent Publication No. US20160279388, entitled “Articulation Systems, Devices, and Methods for Catheters and Other Uses,” published on Sep. 29, 2016, assigned to the assignee of the subject application, the full disclosure of which is incorporated herein by reference.

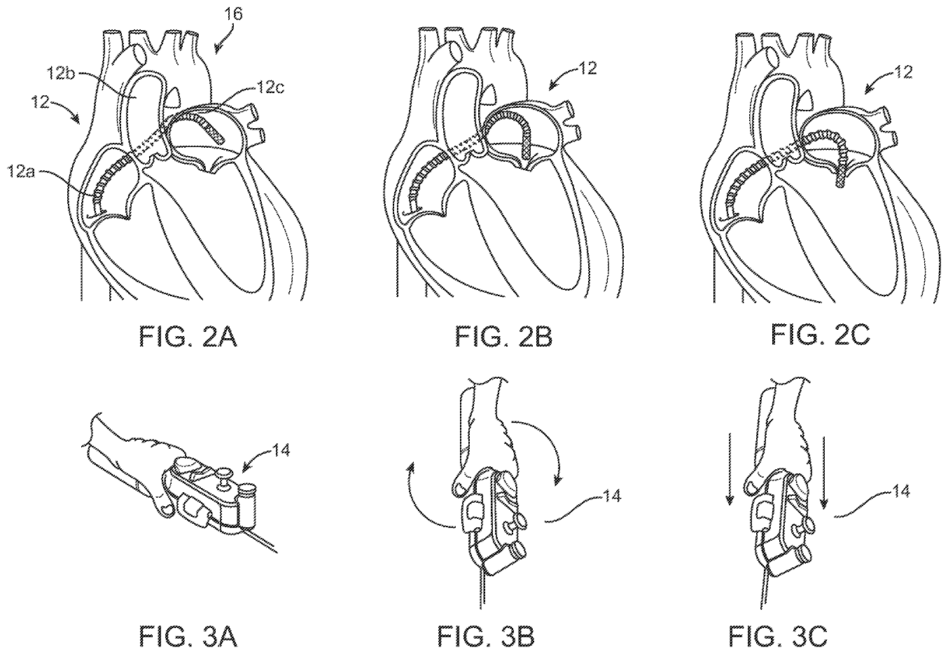

Referring now to FIGS. 2A-3C, devices and methods are shown for controlling movement of the distal end of a multi-segment articulated catheter 12 using a movement command input device 14 in a catheter system similar system 1 (described above). Multi-segment catheter 12 is shown in FIG. 2A extending within a heart 16, and more specifically with a distal portion of the catheter extending up to the heart via the inferior vena cava, with a first, proximal articulatable segment 12a bending within a right atrium of the heart toward a trans-septal access site. A second, intermediate articulatable segment 12b traverses the septum, and a third, distal articulatable segment 12c has some bend inside the left atrium of the heart 16. A tool, such as a prosthetic mitral valve, is supported by the distal segment 12c, and the tool is not in the desired position or orientation for use in the image of FIG. 2A. As shown in FIG. 3A, input device 14 is held by the hand of the user in an orientation that, very roughly, corresponds to the orientation of the tool (typically as the tool is displayed to the user in the display of the image capture system, as described above).

Referring to FIGS. 2A, 2B, 3A, and 3B, to change an orientation of the tool within the heart the user may change an orientation of input device 14, with the schematic illustration showing the input command movement comprising a movement of the housing of the overall input device. The change in orientation can be sensed by sensors supported by the input housing (with the sensors optionally comprising orientation or pose sensors similar to those of smart phones, tablets, game controllers, or the like). In response to this input, the proximal, intermediate, and distal segments 12a, 12b, and 12c of catheter 12 may all change shape so as to produce the commanded change in orientation of the tool. The changes in shapes of the segments will be calculated by a robotic processor of the catheter system, and the user may monitor the implementation of the commanded movement via the image system display. Similarly, as can be understood with reference to FIGS. 2B, 2C, 3B, and 3C, to change a position of the tool within the heart the user may translate input device 14. The commanded change in position can again be sensed and used to calculate changes in shape to the proximal, intermediate, and distal segments 12a, 12b, and 12c of catheter 12 so as to produce the commanded translation of the tool. Note that even a simple change in position or orientation (or both) will often result in changes to shape in multiple articulated segments of the catheter, particularly when the input movement command (and the resulting tool output movement) occur in three dimensional space within the patient.

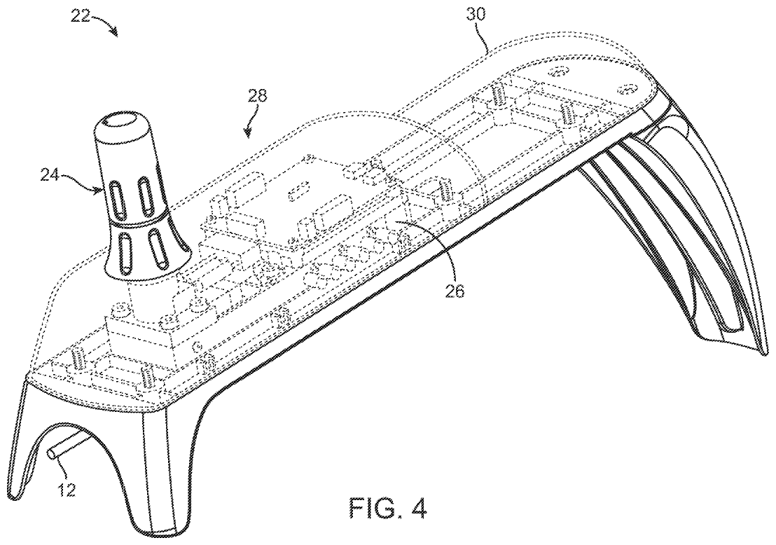

Referring to FIG. 4, an exemplary articulated catheter drive system 22 includes a pressurized fluid source 24 coupled to catheter 12 by a manifold 26. The fluid source preferably comprises a receptacle for and associated disposable canister containing a liquid/gas mixture, such as a commercially available nitrous oxide (N2O) canister. Manifold 26 may have a series of valves and pressure sensors, and may optionally include a reservoir of a biocompatible fluid such as saline that can be maintained at pressure by gas from the canister. The valves and reservoir pressure may be controlled by a processor 28, and a housing 30 of drive system 22 may support a user interface configured for inputting of movement commands for the distal portion of the catheter, as more fully explained in co-pending U.S. patent application Ser. No. 15/369,606 (now issued as U.S. Pat. No. 10,525,233), entitled “INPUT AND ARTICULATION SYSTEM FOR CATHETERS AND OTHER USES,” filed on Dec. 5, 2016 (the full disclosure of which is incorporated herein by reference).

Regarding processor 28 and the other data processing components of drive system 22, it should be understood that a variety of data processing architectures may be employed. The processor, pressure or position sensors, and user interface will, taken together, typically include both data processing hardware and software, with the hardware including an input (such as a joystick or the like that is movable relative to housing 30 or some other input base in at least 2 dimensions), an output (such as a sound generator, indicator lights, and/or an image display, and one or more processor board. These components are included in a processor system capable of performing the rigid-body transformations, kinematic analysis, and matrix processing functionality associated with generating the valve commands, along with the appropriate connectors, conductors, wireless telemetry, and the like. The processing capabilities may be centralized in a single processor board, or may be distributed among the various components so that smaller volumes of higher-level data can be transmitted. The processor(s) will often include one or more memory or storage media, and the functionality used to perform the methods described herein will often include software or firmware embodied therein. The software will typically comprise machine-readable programming code or instructions embodied in non-volatile media, and may be arranged in a wide variety of alternative code architectures, varying from a single monolithic code running on a single processor to a large number of specialized subroutines being run in parallel on a number of separate processor sub-units.

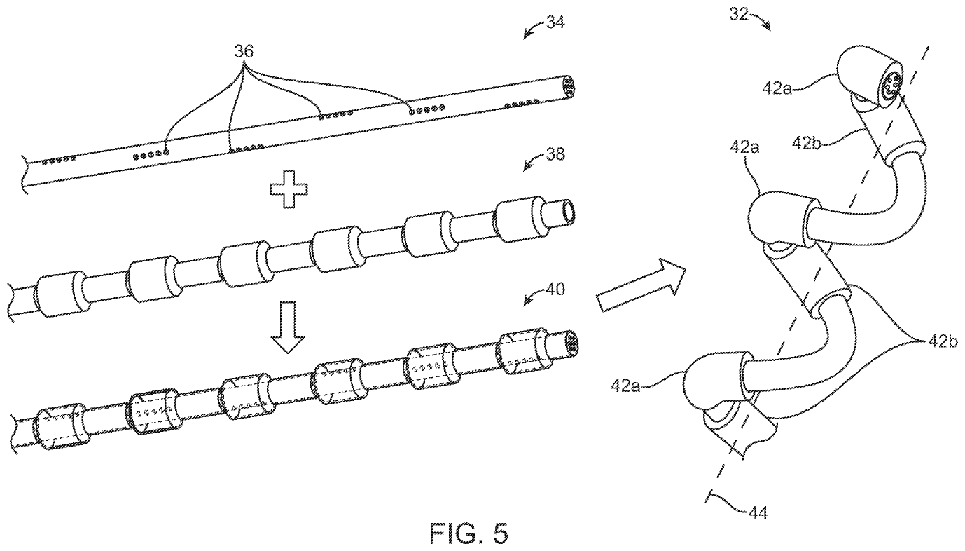

Referring now to FIG. 5, the components of, and fabrication method for production of, an exemplary balloon array assembly, sometimes referred to herein as a balloon string 32, can be understood. A multi-lumen shaft 34 will typically have between 3 and 18 lumens. The shaft can be formed by extrusion with a polymer such as a nylon, a polyurethane, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like. A series of ports 36 are formed between the outer surface of shaft 36 and the lumens, and a continuous balloon tube 38 is slid over the shaft and ports, with the ports being disposed in large profile regions of the tube and the tube being sealed over the shaft along the small profile regions of the tube between ports to form a series of balloons. The balloon tube may be formed using any compliant, non-compliant, or semi-compliant balloon material such as a latex, a silicone, a nylon elastomer, a polyurethane, a nylon, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like, with the large-profile regions preferably being blown sequentially or simultaneously to provide desired hoop strength. The ports can be formed by laser drilling or mechanical skiving of the multi-lumen shaft with a mandrel in the lumens. The shaft balloon assembly 40 can be coiled to a helical balloon array of balloon string 32, with one subset of balloons 42a being aligned along one side of the helical axis 44, another subset of balloons 44b (typically offset from the first set by 120 degrees) aligned along another side, and a third set (shown schematically as deflated) along a third side. Alternative embodiments may have four subsets of balloons arranged in quadrature about axis 44, with 90 degrees between adjacent sets of balloons.

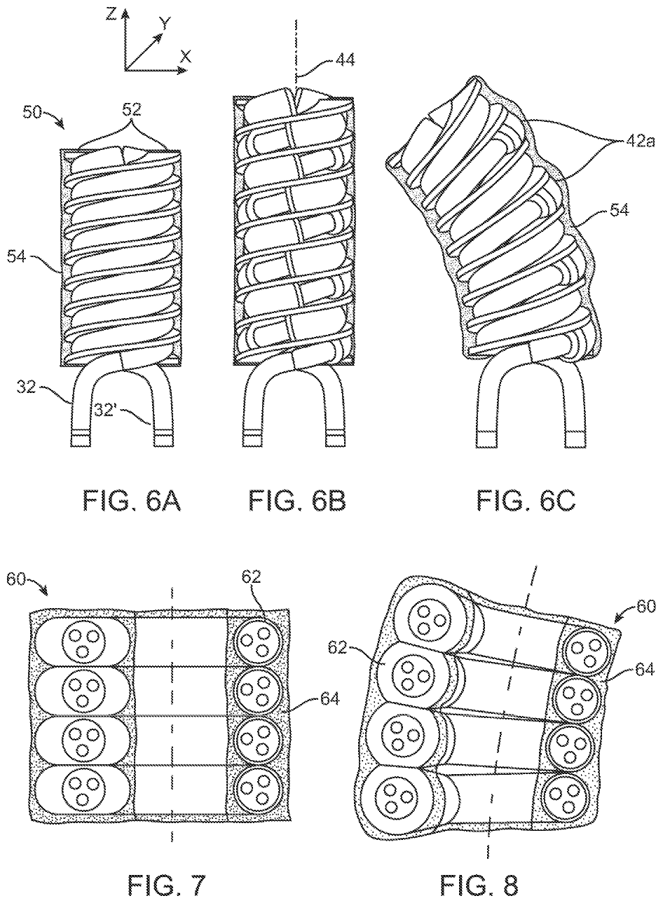

Referring now to FIGS. 6A, 6B, and 6C, an articulated segment assembly 50 has a plurality of helical balloon strings 32, 32′ arranged in a double helix configuration. A pair of flat springs 52 are interleaved between the balloon strings and can help axially compress the assembly and urge deflation of the balloons. As can be understood by a comparison of FIGS. 6A and 6B, inflation of subsets of the balloons surrounding the axis of segment 50 can induce axial elongation of the segment. As can be understood with reference to FIGS. 6A and 6C, selective inflation of a balloon subset 42a offset from the segment axis 44 along a common lateral bending orientation X induces lateral bending of the axis 44 away from the inflated balloons. Variable inflation of three or four subsets of balloons (via three or four channels of a single multi-lumen shaft, for example) can provide control over the articulation of segment 50 in three degrees of freedom, i.e., lateral bending in the +/−X orientation and the +/−Y orientation, and elongation in the +Z orientation. As noted above, each multilumen shaft of the balloon strings 32, 32′ may have more than three channels (with the exemplary shafts having 6 lumens), so that the total balloon array may include a series of independently articulatable segments (each having 3 or 4 dedicated lumens of one of the multi-lumen shafts, for example).

Referring still to FIGS. 6A, 6B, and 6C, articulated segment 50 includes a polymer matrix 54, with some or all of the outer surface of balloon strings 32, 32′ and flat springs 52 that are included in the segment being covered by the matrix. Matrix 54 may comprise, for example, a relatively soft elastomer to accommodate inflation of the balloons and associated articulation of the segment, with the matrix optionally helping to urge the balloons toward an at least nominally deflated state, and to urge the segment toward a straight, minimal length configuration. Advantageously, matrix 54 can maintain overall alignment of the balloon array and springs within the segment despite segment articulation and bending of the segment by environmental forces.

Segment 50 may be assembled by, for example, winding springs 52 together over a mandrel and restraining the springs with open channels between the axially opposed spring surfaces. Balloon strings 32, 32′ can be wrapped over the mandrel in the open channels. The balloons may be fully inflated, partially inflated, nominally inflated (sufficiently inflated to promote engagement of the balloon wall against the opposed surfaces of the adjacent springs without driving the springs significantly wider apart than the diameter of the balloon string between balloons), deflated, or deflated with a vacuum applied to locally flatten and maintain 2 or 4 opposed outwardly protruding pleats or wings of the balloons. The balloons may be pre-folded, gently pre-formed at a moderate temperature to bias the balloons toward a desired fold pattern, or unfolded and constrained by adjacent components of the segment (such as the opposed surfaces of the springs and/or other adjacent structures) urge the balloons toward a consistent deflated shape. When in the desired configuration, the mandrel, balloon strings, and springs can then be dip-coated in a pre-cursor liquid material of polymer matrix 54, with repeated dip-coatings optionally being performed to embed the balloon strings and springs in the matrix material and provide a desired outer coating thickness. Alternatively, matrix 54 can be over-molded onto, sprayed or poured over the balloon strings and springs, or the like. The liquid material can be evened by rotating the coated assembly, by passing the assembly through an aperture, by manually troweling matrix material over the assembly, or the like. Curing of the matrix may be provided by heating (optionally while rotating about the axis), by application of light, by inclusion of a cross-linking agent in the matrix, or the like. The polymer matrix may remain quite soft in some embodiments, optionally having a Shore A durometer hardness of 2-30, typically being 3-25, and optionally being almost gel-like. Other polymer matrix materials may be somewhat harder (and optionally being used in somewhat thinner layers), having Shore A hardness durometers in a range from about 20 to 95, optionally being from about 30 to about 60. Suitable matrix materials comprise elastomeric polyurethane polymers, silicone polymers, latex polymers, polyisoprene polymers, nitrile polymers, plastisol polymers, or the like. Regardless, once the polymer matrix is in the desired configuration, the balloon strings, springs, and matrix can be removed from the mandrel. Optionally, flexible inner and/or outer sheath layers may be added.

Referring now to FIGS. 7 and 8, a simple articulated segment 60 includes a single balloon string 62 supported by a polymer matrix 64 in which the balloon string is embedded. A multilumen shaft of balloon string 62 includes 3 lumens, and the balloons of the balloon string are shown in a nominally inflated state in FIG. 7, so that the opposed major surfaces of most of the balloons of each subset are disposed between and adjacent balloons of that subset on adjacent loops, such that pressure within the subset of balloons causes the balloons to push away from each other (see FIG. 8). Optionally, the balloons of the subset may directly engage each other across much or all of the balloon/balloon force transmission interface, particularly when the balloons are dip-coated when in the nominally inflated state. Alternatively, a layer of matrix 64 may be disposed between some portion or all of the adjacent force-transmission balloon wall surfaces of the subset, for example, if the balloon strings are dip-coated in a deflated state. As can be understood with reference to FIG. 8, inflation of one or more subsets of the balloons may separate adjacent loops of the balloon string between balloons, along the tapering balloon ends, and the like. Elastic elongation of matrix 64 may accommodate some or all of this separation, or the matrix may at least locally detach from the outer surface of the balloon string to accommodate the movement. In some embodiments, localized fracturing of the polymer matrix in areas of high elongation may help to accommodate the pressure-induced articulation, with the overall bulk and shape of the relatively soft matrix material still helping to keep the balloons of the helical balloon array in the desired alignment.

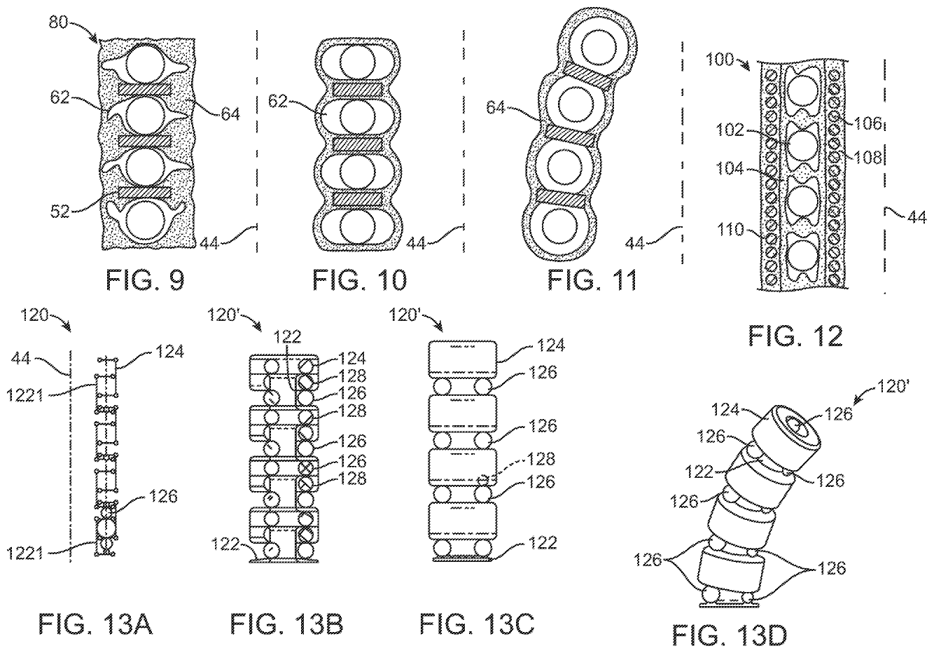

Referring now to FIGS. 9-11, an alternative segment 80 has a single balloon string 62 interleaved with a flat spring 52, and both the balloon string and spring are coated by an elastomeric polymer matrix 64. Shape setting of the balloons may be optionally be omitted, as axial compression of spring 52 can help induce at least rough organization of deflated balloons 62 (as shown in FIG. 9). Local inclusion of some matrix material 64 between the balloon walls and adjacent spring surface (see FIG. 10) may not significantly impact overall force transmission and articulation, particularly where the balloons are generally oriented with major surfaces in apposition, as the pressure force can be transmitted axially through the soft matrix material. Alternatively, the balloons may be nominally inflated during application of the matrix material, as noted above, providing a more direct balloon wall/spring interface (see FIG. 11). As with the other embodiments of segments described herein, flexible (and often axially resilient) radially inner and/or outer sheaths may be included, with the sheaths optionally comprising a coil or braid to provide radial strength and accommodate bending and local axial elongation, such inner and/or outer sheaths often providing a barrier to inhibit release of inflation fluid from the segment should a balloon string leak.

Referring now to FIG. 12, an exemplary segment 100 was fabricated with an intermediate sub-assembly including balloon string 102 embedded in an intermediate matrix 104. An inner sheath is formed radially inward of (and optionally prior to the assembly of) the intermediate sub-assembly by embedding an inner spring 106 within an inner matrix 108. An outer sheath is formed radially outward of (and optionally after assembly of) the intermediate assembly, with the outer sheath including an outer spring 110 and an outer matrix. Note that as in this embodiment, it will often be beneficial for any inner or outer spring to be counterwound relative to the balloon string. First, when the loops of the springs cross the balloons it may help inhibit radial protrusion of the balloons through the coils. Second, it may help to counteract rotational unwinding of the balloon coil structure with balloon inflation, and thereby inhibit non-planar articulation of the segment form inflation of a single balloon subset. Alternative embodiments may benefit from harder matrix materials encompassing the inner or outer springs (or both), from replacing the inner or outer springs (or both) with a braid or eliminating the springs altogether, or the like.

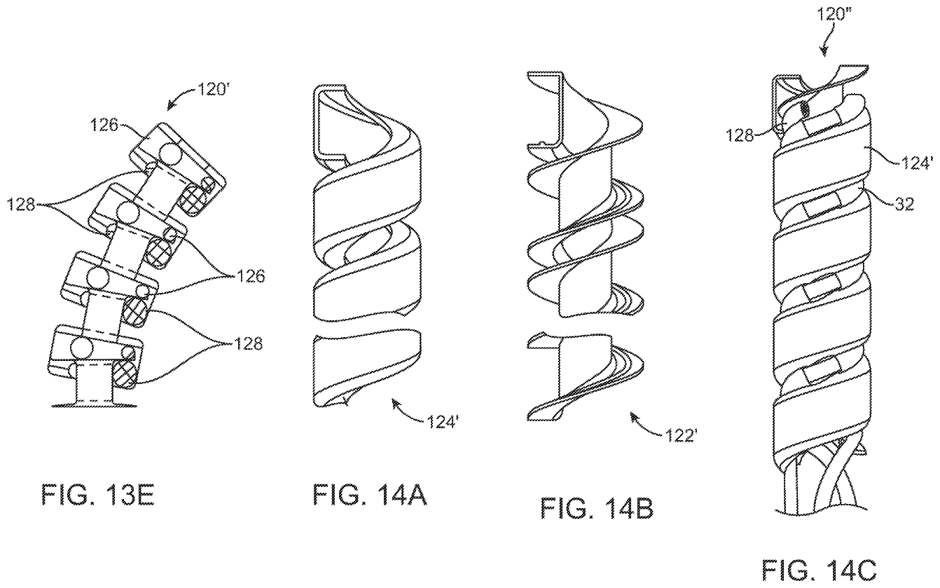

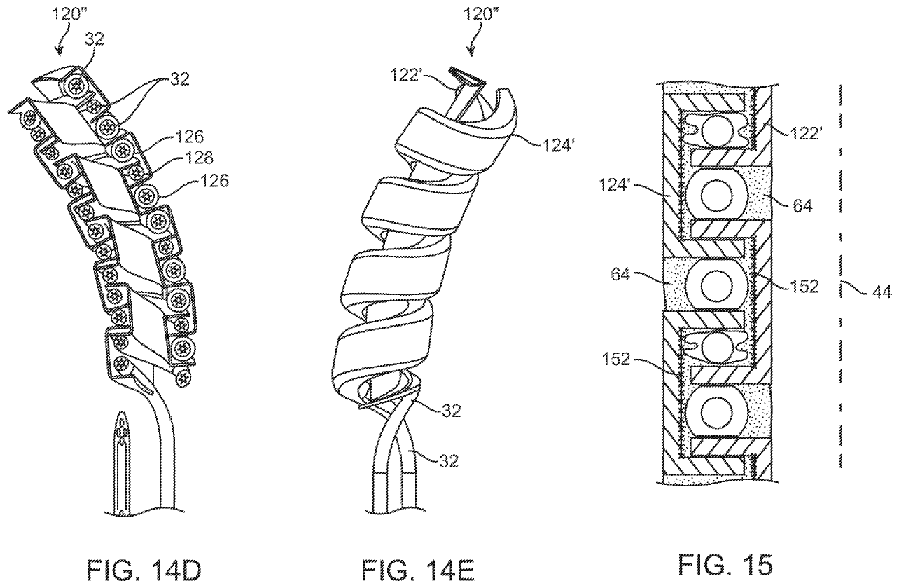

Referring now to FIGS. 13A-14E, alternative segment structures include opposed balloons disposed within channels of segment frames or skeletons to locally axially elongate or contract the frame, thereby laterally bending the frame or changing the axial length of the frame. Referring first to FIG. 13A, a schematically illustrated frame structure 120 includes an axially interleaved set of frame members, with an inner frame 122 having a radially outwardly open channel, and an outer frame 124 having a radially inwardly open channel. The channels are both axially bordered by flanges, and radially bordered (at an inner or outer border of the channel) by a wall extending along the axis. A flange of the inner frame extends into the channel of the outer frame, and a flange of the outer frame extends into the channel of the inner frame. Axial extension balloons 126 can be placed between adjacent flanges of two inner frames or between flanges of two adjacent outer frames; axial retraction balloons 128 can be placed between a flange of an inner frame and an adjacent flange of an outer frame. As more fully explained in US Patent Publication No. US20160279388, entitled “Articulation Systems, Devices, and Methods for Catheters and Other Uses,” published on Sep. 29, 2016 (assigned to the assignee of the subject application and the full disclosure of which is incorporated herein by reference), inflation of a subset of extension balloons 126 along one side of the frame locally extends the axial length of the frame and can bend the frame away from the balloons of the subset. A subset of retraction balloons 128 is mounted in opposition to that local extension, so that inflation of those retraction balloons (with concurrent deflation of the extension balloons) may move the flanges between the balloons in the opposed direction, locally decreasing the length of the frame and bending the axis of the frame toward the inflating retraction balloons. As can be understood with reference to FIGS. 13B-13E, annular frame segments 120′ may have an axially series of ring-shaped inner and outer frames defining the flanges and channels. As shown in FIGS. 14A-14E, helical versions of the frame system may have helical inner and outer frame members 122′, 124′, with extension balloons 126 and retraction balloons 128 being disposed on multiple helical balloon strings extending along the helical channels.

Referring now to FIG. 15, embedding the balloons within the helical frames 122′, 124′ or ring frames described herein within polymer matrix 64 may help maintain alignment of the subsets of balloons despite frame articulation. Articulation performance may be enhanced by the use of soft matrices (with Shore A durometers of 2 to 15), and by inhibiting adhesion at the frame/matrix interface 152 between the axial wall of the frames and the matrix in the channels. Preferably, a slippery interface 152 is provided by a low-friction surface in the channels of the frames between flanges, such as by coating the axial walls with a mold release agent, a PTFE polymer coating or flange material, or the like.

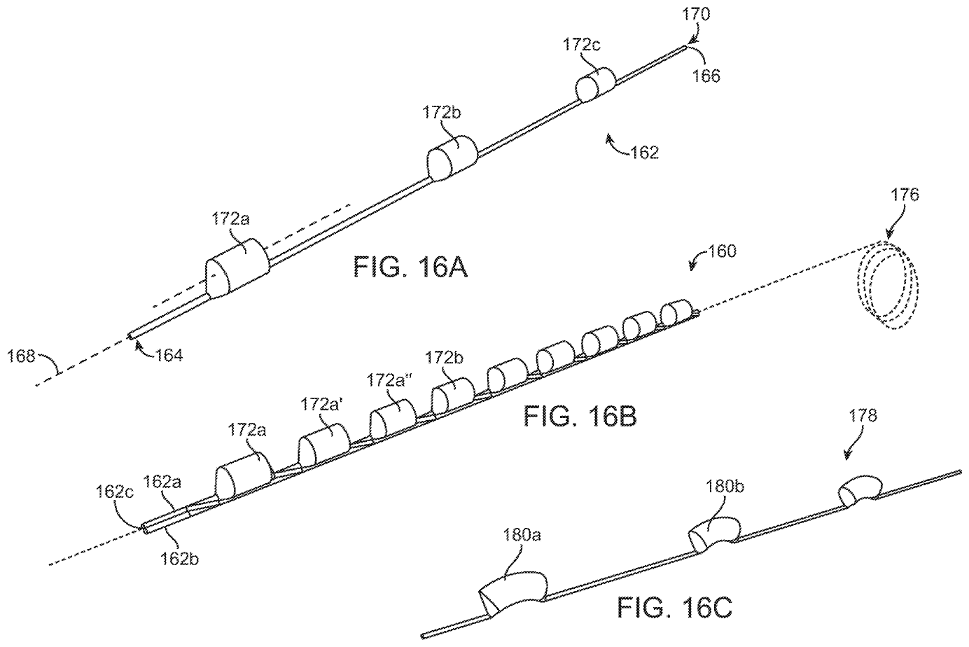

As can be understood with reference to FIGS. 5 and 16A-16C, as an alternative to the multi-channel balloon string 32 used in any of the articulated segments described herein, a balloon string bundle 160 can be assembled by twisting together a group of single-channel balloon stings 162. Single channel balloon string 162 generally comprises tube of polymer material having a first end 164 and a second end 166 extending along an axis 168. A lumen 170 extends along the axis, and the material of the tube is suitable for blowing a series of axially separated balloons 172a, 172b, 172c, . . . . The balloons 172 have a common balloon axis 174 that is offset from axis 168, and may be separated axially by a distance corresponding to a helical arc length of a full loop of the helical balloon array, so that the balloons of a particular string will be substantially aligned along one side (and hence one lateral bending orientation) of the segment.

As can be understood with reference to FIG. 16B, bundle 160 will often be formed using three single channel strings 162a, 162b, and 162c, or four single channel strings (though alternative systems may use other numbers, including only one single channel string, 5, 6, or 8 single channel strings, or the like. The single channel balloon strings within bundle 160 may be axially offset, so that the first balloons 172a, 172a′, 172a″ of strings 162a, 162b, 162c, respectively, are evenly separated from each other, and so that balloon 172a″ is similarly evenly spaced between balloon 172a′ and balloon 172b. The tube material and individual balloon sizes may be similar to those of the individual balloons of the embodiments described above, with the single channel balloons typically comprising a semi-compliant or non-compliant balloon material and being blown sequentially or in batches.

So that the offset axes of the balloons are aligned along a common side (and offset axis) relative to the tubes, the balloon tubes are twisted, with the twist primarily, substantially entirely, or entirely being disposed between the cylindrical portion of the balloons and or between the balloon ends. When three single channel strings are included in the bundle, the tubes may be twisted by about 120 degrees between each balloon and the next; when four channel strings will be included, the tubes may be twisted by about 90 degrees between each balloon and the next. The twisted balloon strings may be heat-set to maintain the alignment of the balloons in the bundle, and/or the strings can be bonded together using an adhesive, heat, laser, or RF welding, or the like. Bundle 160 can be wrapped over a mandrel or otherwise formed into a helical shape 176, with the offset balloon axes often being radially outward of the tube axes or radially inward of the tube axes. Note that the balloon subsets will often be in communication in series via the discontinuous tubes from which they were blown, so that the adhesive or other fluid-tight sealing of the balloons along the array can be avoided. Optionally, as seen in pre-bent balloon string 178 of FIG. 16C, the balloons may be formed as pre-bent offset balloons 180a, 180b, . . . , having an axial curvature corresponding to that of helix 176 so as to facilitate uniform balloon organization, folding, and axial balloon/frame engagement, with the pre-bent balloon shape being defined by clamshell molds or the like. Still other alternative embodiments may also be provided, for example, in which the balloons are blown from a multi-lumen extrusion, with the extrusion optionally having a cross-section in which each of three or four lumens has a surrounding lobe (optionally with indentations therebetween), and with the extrusion being twisted between offset balloons (resulting in an integral structure similar in overall appearance to bundle 160).

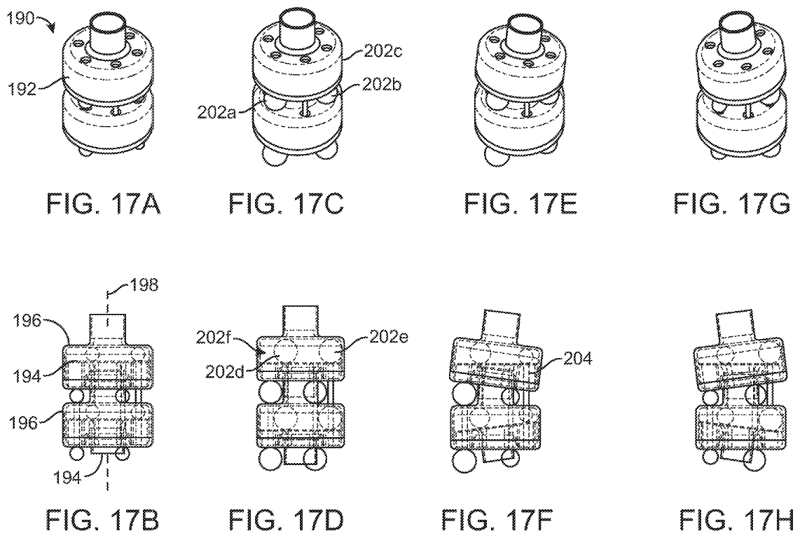

Referring now to FIGS. 17A-17H, an axial string ring frame segment architecture 190 includes a frame 192 having axially alternating inner ring frames 194 and outer ring frames 196. Each ring frame includes an inner tubular portion; a pair is formed by affixing an inner tube portion of an outer ring to an inner tube portion of an inner ring, giving the pair an interlocking S-shaped axisymmetric cross section, with a flange of the inner ring frame from a first pair extending radially outwardly from the inner tube portions into a channel within the outer ring frame of an adjacent pair. Balloons disposed between at least one of the channel-defining flanges of an outer ring and the flange of the inner ring disposed therebetween, and/or between adjacent channel-defining flanges of adjacent pairs, can alter an axial bend characteristic of the ring frame relative to axis 198. The flanges can include relatively rigid polymer or metal structures as the bending occurs between adjacent pairs of ring frames, the frames typically comprising a stainless steel. The ring frames of the pair may be bonded together such as with an adhesive, laser welded, press fit, spot welded, or the like.

Referring still to FIGS. 17A-17H, two sets of axial balloon strings 202a, 202b, 202c, and 202d, 202e, 202f are included in segments 190. The first set of three balloon strings 202a, 202b, 202c provides balloons disposed between outer frame flanges of adjacent pairs, with the three balloons being distributed circumferentially about the axis of the segment with about 120 degrees of separation. The second set of three balloon strings 202d, 202e, 202f are similarly circumferentially distributed and interspersed between the strings of the first set. The balloons of the second set are disposed axially within a channel of an outer frame between an outer frame flange and the inner frame flange. An annular and axially compressible bias member 204 is disposed between the inner frame flange and the other outer frame flange of the channel, so that the bias member is compressed (and the inner frame moves axially) with inflation of the balloons of the second set. Each balloon string comprises an axial series of balloons separated by balloon tube material, with the tubes passing through axial apertures in the flanges of the inner and outer frames. Each balloon proximal of the distal-most balloon of the string has first and second opposed ends, and each end has an associated port providing fluid communication between the balloon and the adjacent tube lumen. The balloon ends engage the flanges surrounding the apertures. Advantageously, the separation distance between the flanges that are spanned by the tubes remains fixed despite the change in inflation state and axial length of the balloon. Changes in inflation of a complete set of balloons can change an axial length of the frame (as can be understood by comparing FIGS. 17A, 17B, 17C, and 17D). Changes in inflation state of the balloons of a set can change a bend orientation and magnitude of the frame axis, as can be understood by comparison of FIGS. 17E, 17F, 17G, and 17H.

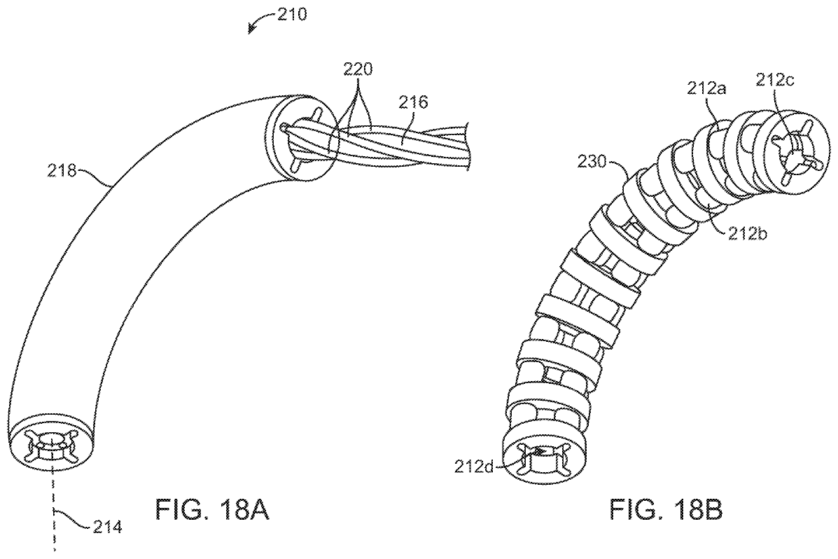

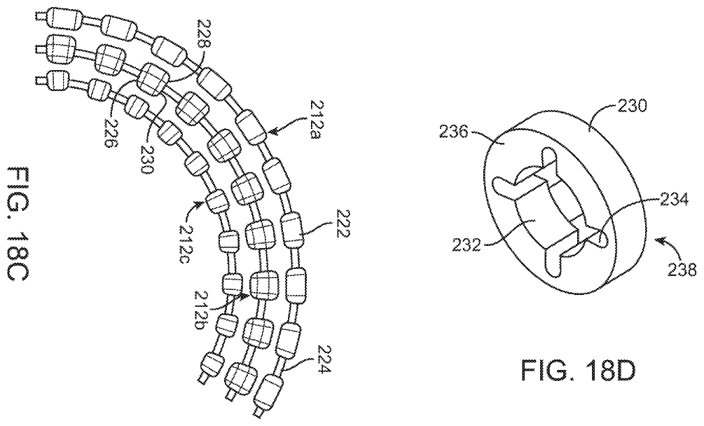

Referring now to FIGS. 18A-18D, a simplified axial balloon string articulated segment 210 includes four axial balloon strings 212a, 212b, 212c, 212d circumferentially separated about a segment axis 214. Balloon strings 212 extend parallel to axis 214 and are disposed radially between a laterally flexible inner sheath 216 and a laterally flexible outer sheath 218. The inner and outer sheaths may have relatively high radial strength and accommodate local axial elongation to facilitate articulation, with the sheaths optionally including an elastomeric polymer and circumferentially oriented windings of a high tensile-strength polymer or metal, such as axially separated steel coil loops, coiled or braided monofilament fibers, or the like. For articulation of segments or other structures distal of segment 210, a plurality of fluid transmission tubes 220 may extend axially within outer sheath 218, the transmission tubes optionally winding helically about axis 214 so as to accommodate localized elongation and lateral bending. Transmission tubes may not have balloons associated with them within segment 210, but may be in fluid communication with balloons of segments distal to segment 210.

Balloon strings 212 each include an axial series of balloons 222 coupled together in series by a tube of balloon material 224. Each balloon 222 generally includes a proximal end 226 and a distal end 228, with the tube interfacing with each balloon end at an associated port. The balloons between the ends may be cylindrical if not constrained, and/or may have an elongate cross-section when in use (such as by radially constraining the balloon between inner and outer sheaths 216, 218).

An axial series of annuli 230 are included in segment 210, with each annulus including a central working channel 232 for receiving the inner sheath 216 and circumferentially separated channels 234 for receiving tubes 224 of balloon strings 212. The annulus has a proximal face 236 and a distal face 238, and the balloons 222 are disposed between adjacent annuli so that the balloon ends 226, 228 engage the opposed faces and push the annuli apart when inflated, thereby bending axis 214. Note that in this embodiment, the balloon tube channels 234 are open radially inwardly to the working lumen to facilitate assembly. Alternative embodiments may include apertures or channels that extend radially outwardly to the outer circumference of the annulus.

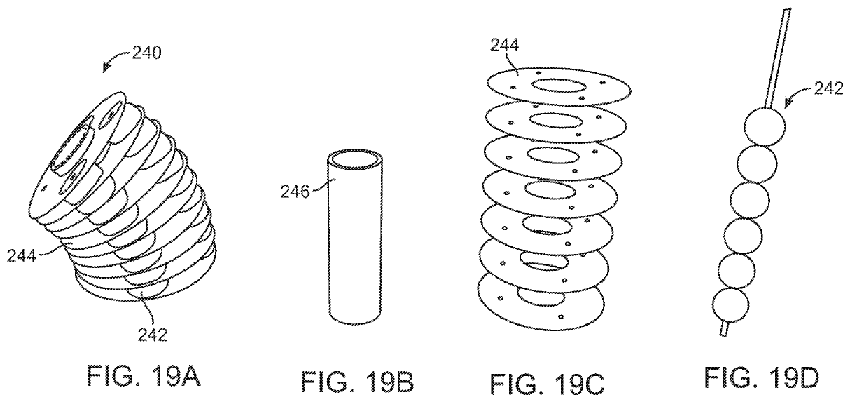

Referring now to FIGS. 19A-19D, a related embodiment of articulated segment 240 includes axial balloon strings 242, with balloons that are disposed between annuli or flat rings 244. The tubes of balloon strings 242 pass through apertures in rings 244, and the rings are bonded to a laterally flexible inner sheath 246 so that in at least some, most, or all of the range of motion of the segment, the balloons have an axial thickness that is less than one a radial width of the balloon and/or a circumferential length of the balloon. Low aspect ratio balloons (those having thicknesses that are less than the width or length or both) and balloons that increase in balloon/ring engagement area throughout a working range of increasing axial compression may have advantages for segment stability, promoting of uniform bending along the axis of the segment, total force load capacity, and/or positioning accuracy.

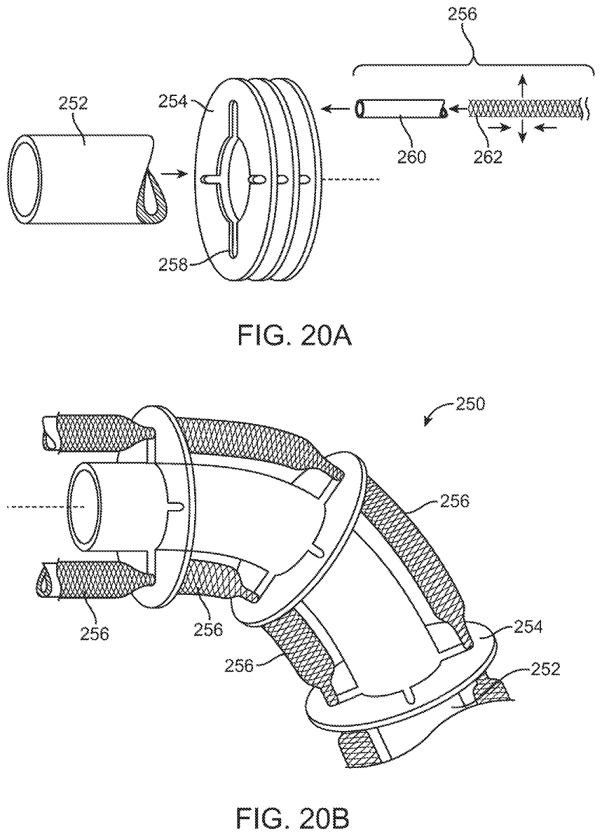

Referring now to FIGS. 20A and 20B, another alternative axial array string articulated segment 250 is shown in an exploded view and a perspective view, respectively. As generally described above, an inner sheath extends axially through annular bodies 254, and fluid expandable bodies 256 extend axially through circumferentially separated channels 258 through the bodies. In this embodiment, fluid-expandable bodies 256 are formed using a resilient bladder 260 within a braided filament tube 262. The braided tube can be shortened to facilitate insertion of the bladder, and the braided filaments can reinforce the bladder to help withstand desired inflation pressures. The relative axial lengths of the expandable body portions and the spacing of the annular bodies can be adjusted during assembly, with sequential bonding of these components together to provide the desired performance.

When expandable bodies 256 are in a large aspect ratio configuration (with greater lengths between annular bodies than diameters), radial expansion of the bladder will often induce tension in the filaments of the braid, generating a net axial tension in the expandable body that can pull the adjacent annular bodies toward each other. Note that the array string, including an axial series of expandable bodies, can be formed by arranging, positioning, and bonding the components, and optionally without blowing individual pre-formed balloons. Relatively small diameter, non-expandable inflation fluid lumens of the strings between expandable bodies may be radially constrained by the surfaces bordering channels 258 of annular bodies 254, and/or by adhesive or other bonds locally extending around the fiber braiding. Ports between the expandable bodies may be similarly formed, and the reduced diameters of the expandable bodies near the annular bodies may facilitate axial bending of the strings, while separating each axial string into multiple expandable bodies may promote even bending along the articulated segment. The axial tension-inducing capabilities of the expandable bodies 256 are well described with reference to McKibben muscle actuators and the various variants that may be used in alternative embodiments of the articulated segments described herein, including actuators having fibers embedded within the bladder material, actuators having knit (rather than braided) fibers, and the like. Alternative embodiments may make use of array strings having similar bladder and fiber expandable body arrays for use as compressive structures as described above. For example, the individual expandable bodies may have low aspect ratios (optionally being larger in diameter than they are long) and the ends of the expandable bodies may apply compressive forces against the adjacent annular bodies so as to locally push the annular bodies apart and curve the segment axis away from the inflating string.

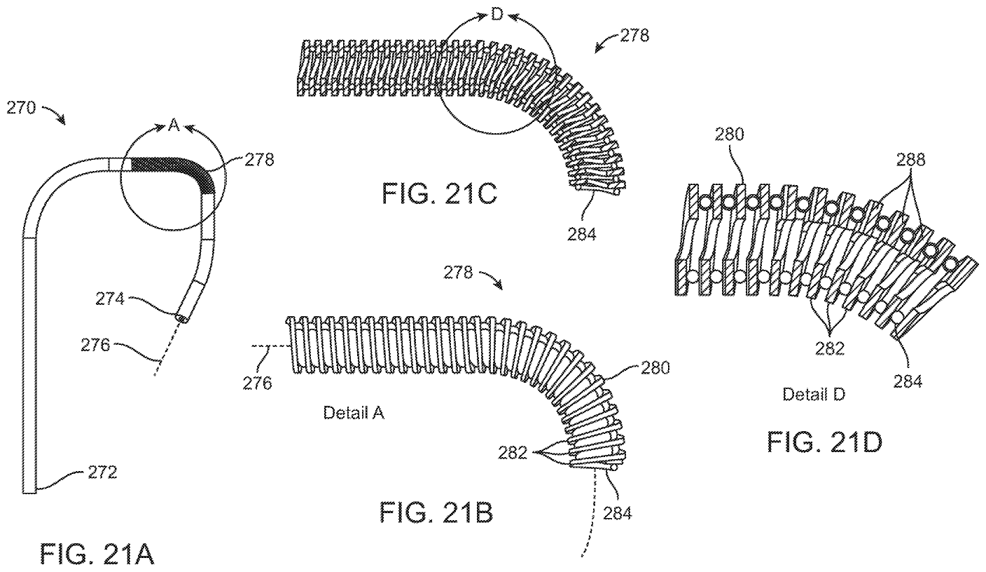

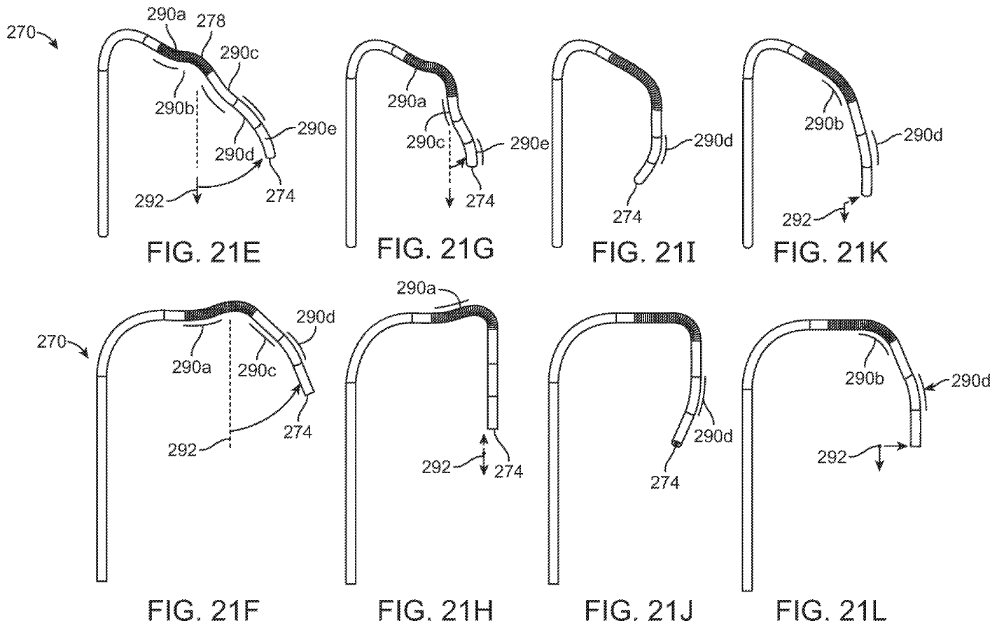

Referring now to FIGS. 21A-21D, an alternative articulated catheter 270 has a proximal end 272 and distal end 274 with an axis 276 extending therebetween. One or more pre-bent articulated segment(s) 278 are between the ends, with the exemplary series of bent segments each having a single degree of freedom, and being arranged to facilitate re-positioning and re-orienting distal end 274 throughout a range of motion bordered by a relaxed, nominal pose of the catheter.

First describing the components providing the axial pre-bend for segment 278 as shown in FIGS. 21B-21D, segment 278 includes a frame having a helical coil 280, with the coil having a plurality of loops 282. As can be seen in the side view of FIG. 21B and the cross-section of FIG. 21C, interleaved between loops 282 of coil 280 is a helical balloon string 284. The balloon string may take the form of any of the balloon strings or bundles described herein, with the exemplary balloon string comprising a single lumen with balloons aligned along the outside of the bend, so that inflation of the balloons reversibly increases an angle and decreases a radius of curvature of the bend. Alternative embodiments may have a plurality of subsets of balloons circumferentially offset from each other by 90 or 120 degrees so as to facilitate 2D adjustment to the orientation and curvature of the bend, or 3 or 4 circumferentially offset balloon subsets so as to allow articulation about a range of motion centered at the pre-bent configuration.

To define the pre-bend along segment 278, a series of axial spacers 288 are disposed between the balloons of string 284 and the loops 282 of coil 280. The spacers may comprise polymer or metal, and are selectively positioned along the outer radius of the bend, locally increasing the separation between loops 282. Spacers 288 may comprise arc segments of a helical or annulus, such as by cutting segments of a spring coil or washer into desired angled portions. The spacers may have a constant axial thickness for simplicity, or may taper circumferentially down from a thicker balloon-engaging central portion to a thinner cross-section near the arc-ends. Spacers 288 may be held in place by adhesive bonding, laser welding, or the like, and some or all of the components of the assembly may be embedded in a polymer matrix by dip-coating or spaying, as generally described above. Segment 278 remains laterally flexible, and the axis along the segment can be restrained in a straightened configuration within a delivery sheath or when the catheter is advanced over a sufficiently stiff guidewire, for example, by resilient deformation of coil 280 so as to separate loops 282 opposite spacers 288.

Referring now to FIGS. 21E-21L, an axial series of single degree-of-freedom segments 290a, 290b, 290c, 290d, and 290e, at least one (but in this case all) segments are a pre-bent segment 278, allows articulation of distal end 274 from a nominal or relaxed position and orientation 292 to a desired position and orientation. With catheter 270 in the configuration of FIGS. 21E and 21F (shown in a perspective view and front view, respectively), pre-bent segments 290a, 290c, 290d, and 290e have pre-configured both the position and orientation of end 274, with the segments here shown in their relaxed position and spacers causing the preliminary curves of the five segments. In the subsequent illustrations of these structures, balloons induce more or less curvature deepening on the joint. Some joints pass through a straight configuration at a mid-point of their associated ranges of motion, which may have benefits for particular joints and in certain situations, for example, including segments 290a, c, d, e. In the configuration of FIGS. 21G and 21H, selective articulation of segments 290a, 290c, and 290e induce translation of end 274, but with an orientation other segments being the same as the nominal configuration. For 21G & H, segments 290b and 290d are articulated so as to cause the tip 274 to be parallel to a proximal portion of the catheter shaft; 290c & 290e are still bent as originally set but in opposed directions.

As shown in FIGS. 21I and 21J, selective articulation of all segments induces a translation and significant re-orientation. In these configurations, segments 290a, 290c, 290d, and 290e are configured in the mid-point of their range of motion, with segments 290a, 290c, 290e in a straight segment configuration. Comparing FIGS. 21G/H with FIGS. 21K/L illustrate how coordinated articulation of several segments (such as segments 290a, 290b, and 290c) can be used to maintain an orientation of end 274 while inducing translation of the end. In the configuration of FIGS. 21K/L, only segment 290b may be articulated from the nominal configuration. Note that other optional combinations of the embodiments described herein may be provided. For example, the single DOF segments described herein can be used with multi DOF segments to maximize function for specific targeted tissues and therapies.

While the exemplary embodiment have been described in some detail for clarity of understanding and by way of example, a variety of modifications, changes, and adaptations of the structures and methods described herein will be obvious to those of skill in the art. Hence, the scope of the present invention is limited solely by the claims attached hereto.

What is claimed is:

1. An articulation system comprising:
an elongate flexible structure having a proximal end and a distal end with an axis therebetween, wherein at least one articulated segment extends along the axis;
an array of fluid-expandable bodies distributed along the at least one articulated segment, the array comprising:
a first array string including a first subset of fluid-expandable bodies offset from the axis so as to laterally articulate the structure with a first lateral bend when inflated, and a first inflation tube having a first lumen in fluid communication with the expandable bodies of the first subset; and
a second array string separate from the first string along the at least one articulated segment and including a second subset of fluid-expandable bodies offset from the axis so as to laterally deflect the structure with a second bend offset from the first bend when inflated, and a second inflation tube having a second lumen in fluid communication with the bodies of the second subset.

2. The articulation system of claim 1, wherein the expandable bodies of the first subset are formed from material of the first tube so that inflation fluid flowing to the first subset flows from the first lumen to the first expandable body of the first subset through a first port, the first body being in fluid communication to the other expandable bodies via a second port of the first body, and the other expandable bodies being in fluid communication, in series, to each other via other associated ports, the ports being substantially inexpansible.

3. The articulation system of claim 1, wherein the first and second inflation tubes are twisted together in an inflation tube bundle.

4. The articulation system of claim 3, wherein the expandable bodies comprise offset balloons and the twisting of the tubes being disposed primarily between the balloons so that the balloons are in alignment along a side of the bundle.

5. The articulation system of claim 1, wherein the first and second array strings are circumferentially separated along a first articulated segment.

6. The articulation system of claim 5, wherein the first articulated segment has a working lumen extending along the axis, and further comprising a plurality of annular bodies distributed along the axis, each annular body being disposed around the working lumen, an axial support maintaining separation between adjacent annular bodies, the annular bodies having circumferentially offset channels receiving the tubes of the array strings axially therethrough, the array strings engaging the annular bodies so that axial articulation forces induced by inflation of the expandable bodies are transmitted to the annular bodies to induce curved articulation along the first segment.

7. The articulation system of claim 6, wherein the structure includes a second articulated segment axially offset from the first articulated segment, the second articulated segment having a plurality of axially separated annular bodies and a plurality of array strings configured to imposed articulation forces thereto so as to facilitate curved articulation of the second segment independent of the first segment.

8. The articulation system of claim 6, wherein the expandable bodies comprise balloons disposed between adjacent annular bodies, the balloons having opposed ends adjacent inflation fluid ports, the balloon ends applying compressive engagement forces against the adjacent annular bodies so as to urge the adjacent annular bodies apart when the balloons are inflated.

9. The articulation system of claim 6, wherein the expandable bodies have ends affixed to adjacent annular bodies and include radially expandable bladders and fibers extending circumferentially about the bladders so as to be loaded in tension and urge the adjacent annular bodies together when the expandable bodies are inflated.

10. The articulated system of claim 1, wherein the first and second subsets are axially separated and disposed along first and second axial articulation segments, respectively.

11. The articulated system of claim 1, wherein the axis along the first articulation segment defines a bend when the structure is in a relaxed configuration, the first articulation segment being deformable toward an axially straight configuration for insertion into a patient.

12. The articulation system of claim 11, wherein variable inflation of the expandable bodies variably articulates the first segment within a range of motion from the bend with a first degree of freedom, and wherein a plurality of other articulation segments define associated axial bends when in the relaxed configuration and are articulatable from the associated bends with associated degrees of freedom, the degrees of freedom arranged so as to allow independent translation and rotation of the structure.

13. The articulation system of claim 11, wherein the first articulated segment comprises a helical coil spring having an axial series of loops with axial spaces therebetween, the expandable bodies of the first subset disposed within the spaces of the spring, further comprising an asymmetrical series of spacers disposed between the loops and expandable bodies so as to urge the first articulated segment toward the bend when the structure is in the relaxed configuration.

14. The articulation system of claim 1, wherein the first and second subsets each include three or more fluid expandable bodies, and wherein a third array string having a third subset of fluid expandable bodies is disposed along the at least one articulated segment so as to articulate the structure laterally along three lateral bending orientations.

15. The articulation system of claim 1, wherein the structure comprises a frame and a polymer matrix, the array strings embedded in the polymer matrix within the frame so as to maintain alignment of the subsets of expandable bodies relative to the axis.

16. An articulation system comprising:
an elongate flexible structure having a proximal end and a distal end with an axis therebetween, wherein an articulated segment extends along the axis;
a first subset of balloons offset from the axis so that a first inflation fluid pressure within the first subset of balloons laterally deflects the body with a first lateral bending orientation, the first subset of balloons including a first balloon, a last balloon, and a plurality of intermediate balloons;
a first lumen disposed within the flexible structure, wherein the first lumen extends distally to a first port of the first balloon of the first subset, and from a second port of the first balloon of the first subset to an adjacent port of an intermediate balloon of the first subset, the first lumen sequentially connecting the balloons of the first subset such that the first inflation fluid pressure within the last balloon of the first subset is transmitted by fluid communication through the first balloon of the first subset and the intermediate balloons of the first subset; and
a second subset of balloons offset from the axis so that inflation fluid pressure within the second subset of balloons laterally deflects the body along a second lateral bending orientation, the second subset of balloons including a first balloon, a last balloon, and a plurality of intermediate balloons; and
a second lumen disposed within the body, the second lumen being discontinuous such that the second lumen extends distally to a first port of the first balloon of the second subset, and extends from a second port of the first balloon of the second subset to an adjacent port of an intermediate balloon of the second subset, the second lumen sequentially connecting the balloons of the second subset such that the second inflation fluid pressure within the last balloon of the second subset is transmitted by fluid communication through the first balloon of the second subset and the intermediate balloons of the second subset.

17. An articulation system comprising:
an elongate structure having a proximal end and a distal end with an axis therebetween, wherein a first articulated segment extends along the axis;
an array of fluid-expandable bodies distributed along the first articulated segment, the array comprising a first subset of fluid-expandable bodies offset from the axis so as to laterally articulate the structure along a first lateral bending orientation; and
wherein the axis along the first segment defines a bend when the structure is in a relaxed configuration, the first articulation segment being resiliently deformable toward an axially straight configuration for insertion into a patient, and
wherein variable inflation of the first subset variably articulates the first segment in a range of motion from the bend.

18. The articulation system of claim 17, wherein a second articulated segment extends along the axis, and wherein the array comprises a second subset of fluid-expandable bodies disposed along the second segment and offset from the axis so as to laterally deflect the structure along a second lateral bending orientation.

19. An articulation system comprising:
an elongate structure having a proximal end and a distal end with an axis therebetween, wherein first, second, and third articulated segments extend along the axis;
an array of fluid-expandable bodies distributed along the first, second, and third articulated segment, the array comprising:
a first subset of fluid-expandable bodies offset from the axis so as to laterally articulate the first articulated segment along a first lateral bending orientation; and
a second subset of fluid-expandable bodies offset from the axis so as to laterally deflect the second articulated segment along a second lateral bending orientation;
a third subset of fluid-expandable bodies offset from the axis so as to laterally deflect the third articulated segment; and
wherein variable inflation of the array variably articulates the elongate structure in at least three degrees of freedom.

20. The articulation system of claim 19, wherein the array comprises three or more subsets along three or more associated segments, each segment having a single associated subset of the array so as to provide lateral bending in a single associated bending orientation, and wherein the array provides movement of the distal end with from three to six degrees of freedom.
